(19) United States
(12) Patent Application Publication
(10) Pub. No.: US 2018/0289762 A1
(43) Pub. Date: Oct. 11, 2018

(54) COMPOSITIONFOR RIDING THE BODY AND HAIR IRREGULARITIES

(71) Applicant: SEANTEREL LATRICE FUGGINS, Oklahoma city, OK (US)

(72) Inventor: SEANTEREL LATRICE FUGGINS, Oklahoma city, OK (US)

(21) Appl. No.: 15/484,623

(22) Filed: Apr. 11, 2017

(51) Int. Cl. A61K 36/82; A61K 31/01; A61K 36/63; A61K 31/355; A61K 31/047

(52) U.S. Cl. CPC A61K 36/82; A61K 31/01; A61K 31/047; A61K 31/355; A61K 36/63

(57) ABSTRACT

This composition of ingredients allows an alternative choice for the body and skin, and hair, (BUT NOT LIMITED TO), that sets forth the healing process, (phonemically faster than the normal pace).

By combining the key natural ingredients, (BUT NOT LIMITED TO), to be used internally and/or externally to heal the body's irregularities. These key ingredients have been previously claimed in an application (Ser. No. 13/684,503, by Seanterel, under the last name Darby, also known as Truely Re'Vived, Earth, Moon, Sun and Wind, (BUT NOT LIMITED TO).

The consistency of the preparation of the ingredients may vary upon what is being made, (but not limited to); Products, that are made from the basic ingredient/'s listed, (the key ingredients in this composition), either it being made in the form of: pills, powders, liquids, solids, and ect. Product/'s from this basic composition, either being made to be ingested, injected, and/or applied to the body topically and thereof, (BUT NOT LIMITED TO).

To determine what or how much the quantities are being used depends each individual product being made, and thereof, (BUT NOT LIMITED TO).

Each varied composition, changes the effects of it potential to heal different problems of the body, with these verse effects the body is relieved of its ailments in a phenomenal time period. These ingredients such as peppermint, mint tea and others (BUT NOT LIMITED TO).

A vast variety of Additives and or Binding carriers, Stabilizers, Emulsifyers, and any type of resins, and thereof, (BUT NOT LIMITED TO), pertaining to (BUT NOT LIMITED TO);

BINDING CARRIERS such as; talcs and clays any and all known, (such as attapulgite, halloysite and lcaolin). Alginates, algea, agars, gums, gelatins, celllluloses, silica, silica gels, simethicone, salicylates, silicates and silicone resins, (such aspolymethtisilsequioxane); tragacanths; charcoal, tragacanths; charcoal, tragacanths, calcium carbonates and magnesium, zin oxides, pastes, ect; and, thereof, (all others—BUT NOT LIMITED TO).

### Uses and Additives

Pharmaceutical or cosmetic form, and thereof, (BUT NOT LIMITED TO); tablets, capsules, (ANY AND ALL ORAL OR INGESTED OR APPLIED OR INJECTED PROCESS), (but not limited to). ointments, creams, gels, lotions, Any and all Hair care products, (BUT NOT LIMITED TO), waxes, (such as beeswax, BUT NOT LIMITED TO), vitamins, Tea's leaves and Tea extractions, such as peppermint, mint of all kinds, (BUT NOT LIMITED TO), powders, liquid powders, syrups, lip balm, oils, any and all liquids, ect; and thereof, (BUT NOT LIMITED TO), AND

Honey, Beeswax and Olive Oil, (BUT NOT LIMITED TO), Inhibits Growth of Staphylococcus aureus and Candida albicans. Al-Waili, N. S. (2005).

Water/s, ANY and ALL liquids, colorant chemicals or fragrances, and thereof, (BUT NOT LIMITED TO).

The key basic ingredients combined formulated consist of tea high in polyphenolic antioxidants (catechins) extracted using mineral oil, (using food grade mineral oil a non-plant or animal oil); It exerts anti-inflammatory, antithrombotic, antihypertensive. Tocopherols and tocotrienols have an antioxidant function. Vegetable glycerin has a hydrating effect, and olive oil has effects of oxidation.

### REFERENCES CITED (REFERENCE BY)

### US Patent Documents;

U.S. Pat. No. 4,062,785 A Dec. 13, 1977 Roger Keith Nibert

U.S. Pat. No. 5,527,552 A Jun. 18, 1996 Paul H. Todd, Jr.

U.S. Pat. No. 5,102,567 A Apr. 7 1992 Leslie R. Wolf

### OTHER REFERENCES

### Wikipedia, Encyclopedia

Mineral Oil CA2317793 c (Grant) Feb. 12, 2008 Gerard P. Mcnally, Charles E. Ii. Pendley

Mineral Oil U.S. Pat. No. 2,602,049 Jul. 1, 1952 Hill, Mark, L. Olive Oil International Olive council, Olive Oil Times, Health Diaries, and Mayo Clinic

Vitamin E U.S. Pat. No. 6,544,530 B1 (GRANT) Mar. 9, 2000 Doron Friedman Packer L. Weber S U, Rimbach, G.

REFERENCE; Ser. No. 13/684,503/US20140147524A1 SEANTEREL LATRICE DARBY/now FUGGINS

REFERENCE; WO2017142866A1 THE UNIVERSITY OF OKLAHOMA

### DESCRIPTIONS

The consistency of the preparation of the ingredients may vary upon what is being made, (but not limited to); Products, that are made from the basic ingredient/'s listed, (the key ingredients in this composition), either it being made in the form of: pills, powders, liquids, solids, and ect. Product/'s from this basic composition, ether being made to be ingested, injected, and/or applied to the body topically and thereof, (BUT NOT LIMITED TO).

To determine what or how much the quantities are being used depends each individual product being made, and thereof, (BUT NOT LIMITED TO).

Each varied composition, changes the effects of it potential to heal different problems of the body, with these verse effects the body is relieved of its ailments in a phenomenal time period. These ingredients such as peppermint, mint tea and others (BUT NOT LIMITED TO).

A vast variety of Additives and or Binding carriers, Stabilizers, Emulsifyers, and any type of resins, and thereof, (BUT NOT LIMITED TO), pertaining to (BUT NOT LIMITED TO);

BINDING CARRIERS such as; talcs and clays any and all known, (such as attapulgite, halloysite and lcaolin). Alginates, algea, agars, gums, gelatins, celllluloses, silica, silica gels, simethicone, salicylates, silicates and silicone resins, (such aspolymethtisilsequioxane); tragacanths; charcoal, tragacanths; charcoal, tragacanths, calcium carbonates and magnesium, zin oxides, pastes, ect; and, thereof, (all others—BUT NOT LIMITED TO).

### Uses and Additives

Pharmaceutical or cosmetic form, and thereof, (BUT NOT LIMITED TO); tablets, capsules, (ANY AND ALL ORAL OR INGESTED OR APPLIED OR INJECTED PROCESS), (but not limited to). ointments, creams, gels, lotions, Any and all Hair care products, (BUT NOT LIMITED TO), waxes, (such as beeswax, BUT NOT LIMITED TO), vitamins, Tea's leaves and Tea extractions, such as peppermint, mint of all kinds, (BUT NOT LIMITED TO), powders, liquid powders, syrups, lip balm, oils, any and all liquids, ect; and thereof, (BUT NOT LIMITED TO), AND

Honey, Beeswax and Olive Oil, (BUT NOT LIMITED TO), Inhibits Growth of Staphylococcus aureus and Candida albicans. Al-Waili, N. S. (2005).

Water/s, ANY and ALL liquids, colorant chemicals or fragrances, and thereof, (BUT NOT LIMITED TO).

### SUMMARY OF INVENTION

The key ingredients in this composition, (BUT NOT LIMITED TO), heal the body and Hair of its conditions and sets forth the healing processes, (faster than the normal pace). The mentioned key ingredients in this composition, combined produce; anti-inflammatory, antithrombotic, antihypertensive, antioxidant, and hydrating effects, and have phenomenal results especially when other ingredients, (Peppermint tea, BUT NOT LIMITED TO), are added for a greater effect on the compositions healing different irregularities.

REFERENCE; Ser. No. 13/684,503/U520140147524A1 SEANTEREL LATRICE DARBY/FUGGINS

Claim has been researched and documented without my approval;

REFERENCE; WO2017142866A1 THE UNIVERSITY OF OKLAHOMA

1; The key basic ingredients form a composition of riding the body internally and externally of it irregularities from mutant possibilities.

2; The process of usage of this composition rids the body phonemically at a fast pace.

3; The ingredients created are combined and used in many forms for healing.

4: The key ingredients are in equal and/or varied amounts (BUT NOT LIMITED TO), and thereof; Tea/'s—0.04% extracted with mineral oil—0.24% totaling 0.28%, Vitamin E—0.24%, Vegetable glycerin—0.24%, and Olive oil—0.24% (BUT NOT LIMITED TO),
INGREDIENTS AND POSSIBLE MECHANISM OF ACTION
FROM THE WIKIPEDIA, ENCYCLOPEDIA DEFINITIONS

5; Green Tea—(extracted), is an antioxidant ingredients, anti-inflammatory characteristics, and bactericidal activity against S. mutans. REF; WO2010/097643

6; Mineral Oil—(lubricant laxative used 99.9% mixed with tocopherols), food grade a non-plant or animal oil, NON COMEDOGENICAL, (DOES NOT CLOG) REF; U.S. Pat. No. 2,602,099

7; Olive Oil—Anti-inflammatory

8; Vitamin E—A TOCOPHEROL, Antioxidant function, BIOLOGICAL FUNCTIONS, ENZYMATIC ACTIVITIES, REF; AHFS/DRUGS.COM MED FACTS NATURAL PRODUCTS, REF; PARKER L. WEBER S U. RIMBACH G (2001).

9; Vegetable Glycerin—hydrating effects, FOR BURNS, REF; U.S. Pat. No. 6,544,530 B1

10; PROCESS OF EXTRACTION AND COMBINING, By combining the mentioned key ingredients, (BUT NOT LIMITED TO), in this composition as follows, (BUT NOT LIMITED TO); ADDING, Green tea/'S, such as Peppermint tea or oils, (BUT NOT LIMITED TO), combined with Mineral oil, (a Non Plant or Animal oil), (BUT NOT LIMITED TO), heated to the point of extraction, then drained and use of the liquid from the extraction with the rest of the ingredients.

11; PEPPERMINT TEA/or OIL MENTHA PIPERITA, OIL MENTHOL, TISANE; DYSPEPSIA HAS HEALING EFFECTS, ANTIMICROBIAL, ANTIVIRAL ACTIVITIES, STRONG-ANTIOXIDANT, ANTITUMOR-ACTIONS, ANTIALLERGENIC-POTENTIAL, ANALGESIC, ANESTHETIC EFFECTS; ANTIBACTERIAL, ANTIFUNGAL, ANTIVIRAL PROPERTIES, (KILLS SOME TYPES OF BACTERIA, FUNGUS, VIRUSES), SOOTHING AND COOLING EFFECT SO SKIN IRRITATIONS CAUSED BY HIVES, POISON IVY, POISON OAK; REF; (2006) JOHN WILEY AND SONS LTD. REF; UNIVERSITY OF MARYLAND MEDICAL CENTER.

12; In this key composition, (BUT NOT LIMITED TO), combined together with/or without the use of heat, (BUT NOT LIMITED TO).

13; WHEN THE ADDITIVE IS ANOTHER (ANY AND ALL), FORM/'s OF LIQUID SUCH AS WATER A DIFFERENT PRESERVATIVE METHOD IS USED AND THE COMPOSITION MAY REQUIRE REFRIGERATION OR STORED AT COOL DRY TEMPERATURE.

14; WHEN DIFFERENT STORAGE, SUCH AS SPRAYS, ALCOHOLS AND AEROSOLS, DISPENSERS, FOAMS, (ANY AND ALL CONTAINERS), (BUT NOT LIMITED TO), OR APPLICATION METHODS ARE USED OR JOINED WITH A SUBJECT MATTER/ITEM, SUCH AS WET WIPES, TREATED CLOTH, SHEETS, FABRICS ANY AND ALL, SKIN KITS, (BUT NOT LIMITED TO), PAPER (BUT NOT LIMITED TO), DIFFERENT INSTRUCTIONS MAY APPLY, (BUT NOT LIMITED TO).

15; HONEY, BEESWAX, AND OLIVE OIL MIXED INHIBITS GROWTH OF STAPHYLOCOCCUS AUREUS AND CANDIDA ALBICANS, REF; ARCHIVES OF MEDICAL RESEARCH 36 (2013).

16; THE AMALAGAMATION; ADDING ANY AND ALL TYPES OF MATTER SUCH AS POWDERS, TALCS, OILS, OILS DERIVED FROM SEEDS OR FRUITS OR VEGETABLES, ALL FRUIT PEELS AND VEGETABLES, (ANY AND ALL), (BUT NOT LIMITED TO), VITAMINS AND MINERALS, AS IN BIOTIN, OR ANY INGREDIENT ON THE PERIODIC TABLE, AND ALL PHARMACEUTICALS, (BUT NOT LIMITED TO), MILK AND MILKY SUBSTANCES TO THE BASIC INGREDIENTS WILL CHANGE NOT ONLY THE EFFECTS BUT ALSO STORAGE PRESERVATION AND THE CHANGES ON IT'S EFFECTS.
